(19) United States
(12) Patent Application Publication
(10) Pub. No.: US 2009/0281421 A1
(43) Pub. Date: Nov. 12, 2009

(54) SYSTEM AND METHOD FOR TARGETED ACTIVATION OF A PHARMACEUTICAL AGENT WITHIN THE BODY CAVITY THAT IS ACTIVATED BY THE APPLICATION OF ENERGY

(76) Inventors: Jerry A. Culp, Oshtemo, MI (US); Richard F. Huyser, Kalamazoo, MI (US); David S. Goldenberg, Mattawan, MI (US)

Correspondence Address: INTEL. PROP./ RND;STRYKER CORPORATION, 4100 EAST MILHAM AVE., KALMAZOO, MI, 49001-6197, US

(21) Appl. No.: 12/474,357

(22) Filed: May 29, 2009

Related U.S. Application Data
(63) Continuation of application No. PCT/US2007/085922, filed on Nov. 29, 2007.
(60) Provisional application No. 60/867,928, filed on Nov. 30, 2006.

(51) Int. Cl. A61N 5/06; A61B 5/055

(52) U.S. Cl. 600/426; 604/20; 607/88; 600/439

(57) ABSTRACT

A system for selectively triggering an energy activated therapy agent. The system includes a member to which a device for emitting energy is attached. One or more navigation markers are also attached to the member. The position of the navigation markers is tracked to provide an indication of the position of the energy emitting device relative to the target tissue. When the energy emitting device is adjacent the target tissue, it is actuated.

### RELATIONSHIP TO EARLIER FIELD APPLICATIONS

This application is a continuation of PCT App. No. PCT/US2007/085922, filed 29 Nov. 2007 which is a non-provisional of App. No. 60/867,928 filed 30 Nov. 2006 the contents of which are incorporated herein by reference.

### FIELD OF THE INVENTION

This invention is generally related to a system and method for emitting the energy to activate pharmaceutical agents in body tissue. More particularly, this invention is related to a system for emitting the energy for maximizing the activation of the agent in the tissue in which the agent is to be activated while minimizing the activation of the agent in the surrounding tissue.

### BACKGROUND OF THE INVENTION

Pharmaceutical agents have been and are being developed that are activated by the application of energy to the agent. One class of agents are photosensitive agents. A photosensitive agent is a compound that, when light at an appropriate wavelength is applied, provides a therapeutic affect in the body tissue in which the agent is absorbed. For example, some photosensitive agents are employed to reduce, ablate, the tissue in which they are absorbed. Initially, in the procedure in which this type of agent is employed, the agent is introduced into the body of the patient. The agent is given time to be absorbed into the specific tissue targeted for reduction. A light emitted at a specific wavelength is then directed to the target tissue. The agent, when exposed to the light in the presence of oxygen, produces oxygen species that have a cytotoxic effect on the surround cell mass. Thus, the oxygen species accelerates the die off of the cell mass forming the tissue. This cell die off is the reduction of the targeted tissue.

A benefit gained by employing an energy-activated pharmaceutical agent to accomplish a therapeutic effect is that the agent is only supposed to be active in the tissue in which the therapy is desired. The selective activation of the agent is to limit undesirable side effects that can occur if the agent is activated in normal tissue. Also, some energy-activated therapeutic agents offer an improved therapeutic affect than the traditional agents that they replace.

In practice, it sometimes is difficult to precisely control in which tissue a energy-activated agent is activated. This is because when a therapeutic agent is introduced into a region of the body, a fraction of the agent almost always diffuses into the tissue adjacent the tissue which is to be subject to therapy. The light or other energy introduced into the body region almost invariably activates the agent diffused into the normal tissue. One practice employed to eliminate this unwanted activation of the agent is to delay the application of energy until the time of maximum difference of agent retention in the tissue that is the subject of therapy and the surrounding normal tissue. A disadvantage of this protocol is that, while waiting for this time period, some of the agent that could have otherwise offered therapeutic effect diffuses through or metabolizes in the tissue on which the therapy is to be performed. Also, it may be difficult to determine for an individual patient when this maximum difference between agent retention in tissue to be treated and the adjacent normal tissue is present. If the agent is activated too early, the result may be undesirable side effects in the normal tissue. If the agent is activated too late, the beneficial effects of the agent are further lost.

### SUMMARY OF THE INVENTION

This invention is directed to a new and useful system and method for precisely activating an energy-activated therapy agent, pharmaceutical agent, retained in a body region. The system and method of this invention is designed to enhance activation of the agent in the tissue targeted for therapy. This invention is also designed to increase the likelihood that agent activation in nearby normal tissue is minimized, if not eliminated.

In the system and method of this invention, a medical imaging/diagnostic equipment unit such as an ultrasonic unit, a CAT unit, a PET unit, a navigation unit or an MRI unit generates a map of the tissue types in the body region in which the tissue requiring therapy is located. As part of the imaging process, the locations of fiducial markers placed on the patient are also identified.

The clinician views the images of the body region generated from the tissue map. As part of this process, the clinician identifies the tissue on which the therapy is performed. A map of tissue targeted for treatment is generated.

The energy-activated pharmaceutical agent is introduced into the body region at which the tissue to be subject to therapy is located.

If the tissue is located immediately below the skin, an activation unit capable of selectively emitting energy into the body is positioned over the body region, against the skin. For example, if the agent is activated upon the application of photonic energy, light energy, this activation unit may be a light blanket. The light blanket includes a substrate on which a number of light emitting elements (LEEs) are mounted. The light emitting elements (LEEs) are directed to the skin.

The unit also contains components that allow the position of the unit to be tracked by a navigation unit external to the patient. The navigation unit also tracks the position of fiducial markers on the patient. Stored within the memory of the system are data indicating where, within the body region the targeted tissue is located.

Based on the above data, the surgical navigation unit is able to determine the location of each LEE relative to the tissue on which the procedure is performed. These data are transferred to a unit that regulates the actuation of the LEEs. If a particular LEE is positioned over a relatively thick section of tissue targeted for treatment, the LEE is actuated for a relatively long amount of time. This maximizes activation of the agent in this specific target tissue. A LEE positioned over a thinner section of tissue to be treated is actuated for a shorter period of time. This ensures the agent is actuated in the targeted tissue while minimizing the activation of the tissue in adjacent normal tissue. Some LEEs may be positioned over normal tissue. These LEEs are not actuated.

The system and method of this invention thus increases the likelihood that the pharmaceutical agent is activated in the tissue in which the agent has the greatest therapeutic effect while minimizing agent activation in adjacent normal tissue.

In one version of the invention, the imaging equipment generates a data map indicating where undesirable fat tissue located immediately below the skin is located. The clinician and the patient view an image of this map. Based on consultation with the patient, the clinician identifies where the fat is to be removed.

In these versions of the invention, the photosensitive agent is one capable of reducing fat.

### BRIEF DESCRIPTION OF THE DRAWINGS

The invention is pointed out with particularity in the claims. The above and further features and advantages of this invention are better understood from the following Detailed Description taken in conjunction with the accompanying drawings in which:

FIG. 1 is a basic diagram of a system for selectively triggering a photosensitive agent of this invention;

FIG. 2 is a plan view of the distal side of a light blanket of this invention;

FIG. 3 is a cross sectional view of the a section of the light blanket;

FIG. 4 plan view of proximal side of the light blanket;

FIG. 5 is a block diagram of the processor of the system of this invention including some of the major instruction modules executed by the processor during the operation of the system;

FIG. 6 depicts how, in the system and method of this invention, the relevant body region of the patient is surveyed in order to generate a map of the tissue on which the procedure is to be performed;

FIG. 7 is a plan view of a patient showing the fiducial markers on the patient and the ultrasonic unit probe being used to scan the tissue internal to the patient;

FIG. 8 is a cross sectional map of the internal tissue of the patient;

FIG. 9 is a cross sectional view of the subcutaneous tissue internal to the patient wherein the locations of the tissue that is to be subjected to treatment are flagged;

FIG. 10 is a cross sectional view of the patient illustrating the locations of the tissue flagged for treatment, the light blanket disposed over the patient and the plots of the paths of the light that would be emitted by some of the light emitting elements integral with the blanket;

FIG. 11 is a flow chart of the process steps by which it is determined the extent to which each light emitting element (LEE) integral with the light blanket is to be actuated.

FIG. 12 is a diagrammatic two-dimensional depiction of the plotting of a light vector from one of the light emitting elements;

FIG. 13 is a plan view of the distal end of a catheter of the system of this invention;

FIG. 14 is a cross sectional view of a tracker used to monitor the position of the catheter;

FIG. 15 is a cross sectional view illustrating how the tracker is used to determine the location and orientation of the catheter relative to a targeted section of tissue internal to the patient;

FIG. 16 is a diagrammatic view of how an alternative diagnostic unit, here an MRI, is used to generate a map of the tissue internal to the patient;

FIGS. 17A and 17B are, respectively, top and cross sectional views of an alternative fiducial marker of this invention;

FIG. 18 is an alternative diagrammatic side view of an alternative version of the fiducial marker of FIG. 17A;

FIG. 19 is a partial plan view of the distal face of an alternative light blanket of this invention;

FIG. 20 is a partial cross sectional view of a light plate of this invention;

FIG. 21 is a plan view of the strap used to hold the light plate;

FIG. 22 is a cross sectional view of how the strap holds the light plate to a section of the body, here the torso;

FIG. 23 is a side view of a first alternative catheter tip of this invention;

FIG. 24 is a side view of a second alternative catheter tip of this invention;

FIG. 25 is a side view of a third alternative catheter tip of this invention;

FIG. 26 is a side view of a fourth alternative catheter tip of this invention;

FIG. 27 is a side view of a fifth alternative catheter tip of this invention;

FIG. 28 is a block diagram of an alternative processor architecture of the system of this invention.

### DETAILED DESCRIPTION

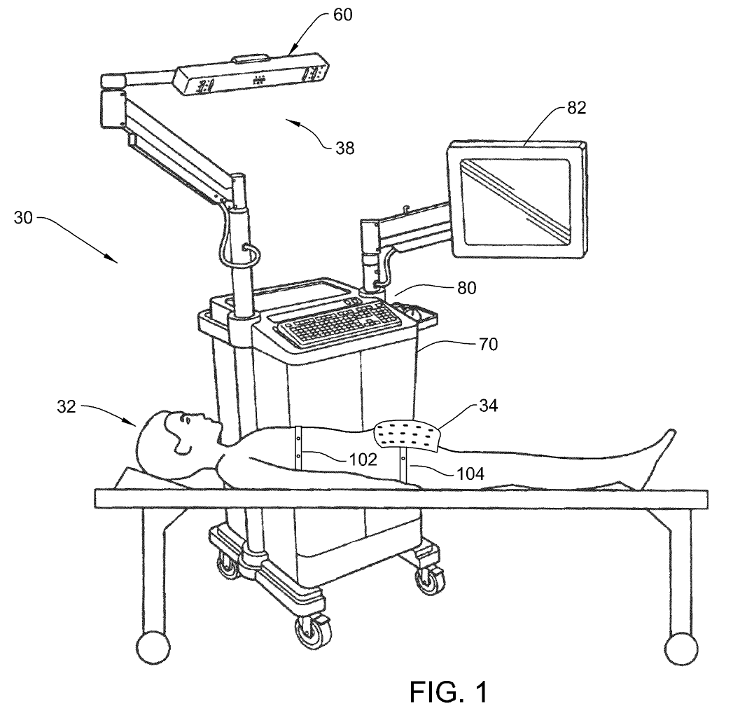

The basic components of a system 30 of this invention for selectively activating an energy-activated pharmaceutical agent are now described by reference to FIG. 1. System 30 is shown being used to trigger an agent that is activated by applying light to the agent. Often this particular class of agent is referred to as a photosensitive agent. As discussed below, this invention is not limited to triggering agents that are activated by the application of photonic energy.

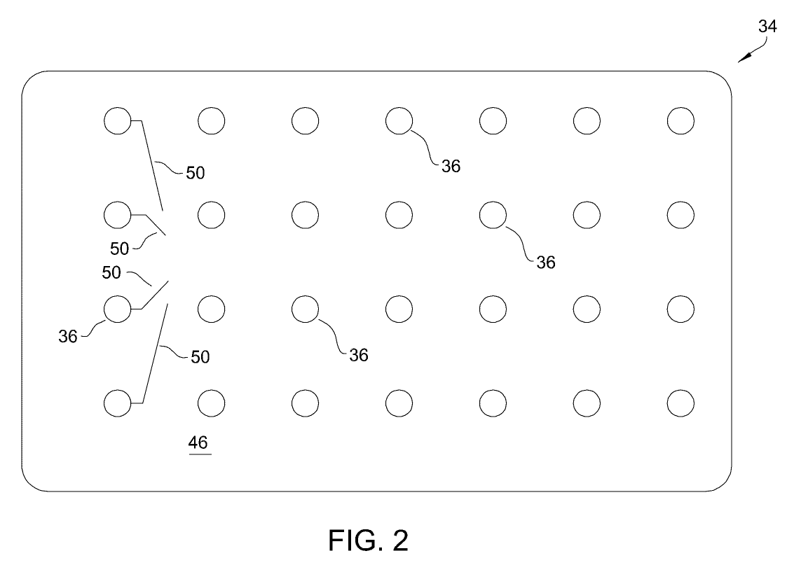

System 30 of this invention is intended to activate a photosensitive agent in a patient 32 immediately below skin level. The system 30 includes a light blanket 34 positioned over the portion of the patient 32 that includes the tissue in which the agent is to be activated. Light blanket 34, as described in detail below, contains a number of individual light emitting elements (LEEs) 36 (FIG. 2). A navigation unit 38 monitors the position of the light blanket 34. More particularly, the navigation unit 38 monitors the position of the light emitting elements 36 relative to the subcutaneous tissue below the light blanket 34.

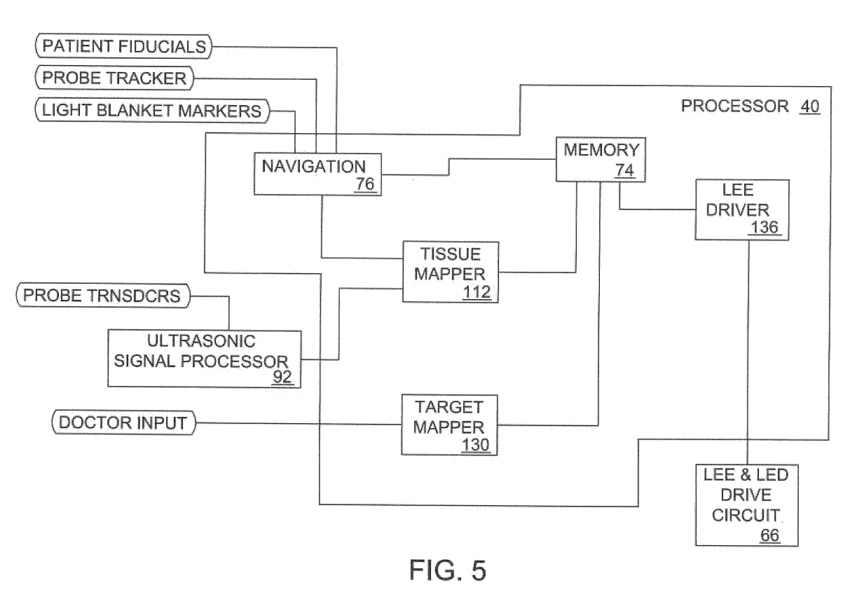

A processor 40, (FIG. 5) also part of system 30, contains a map of the patient's tissue within the relevant body site. This map includes flags indicating in which tissue the photosensitive agent is to be activated. These flags are set by the medical personnel prior to the start of the procedure. Processor 40 also contains data indicating the position and orientation of each light emitting element 36. Based on these data and the map flags, processor 40 selectively actuates each light emitting element 36. If the data indicates the light emitting element 36 is adjacent tissue in which the agent is to be actuated, processor 40 actuates the light emitting element and controls the amount of light so emitted. Alternatively, if the data indicates that sub-cutaneous tissue underlying the light emitting element 36 is not tissue that should be subjected to the procedure, the light emitting element is not actuated.

As seen in FIGS. 2, 3 and 4 the light blanket 34 consists of a substrate 46. In one version of the invention, substrate 46 is formed from non conductive material such as Kapton polyimide film or Mylar polyester film. The substrate 46 is flexible so that when placed over a portion of the body, the substrate is able to conform to the surface of the body. Substrate 46 has two sides; a distal side directed towards the patient and an opposed proximal side. Mounted to the substrate distal side are a number of individually actuated light LEEs 36. Either LEDs or laser diodes may function as the LEEs 36. The LEEs 36 are arranged in a row×column grid. Shown diagrammatically for four (4) of the light emitting elements 36 are conductors 50 over which energization signals are applied to the LEEs 36. Vias, not shown, provide conductive paths through substrate 46. It is further appreciated that when LEDs or laser diodes are employed as LEEs 36, load resistors (not illustrated) are series connected to the individual elements. These resistors are surface mounted to substrate 46. The LEEs 36 are selected so that they emit light at the wavelength necessary to excite the photosensitive agent.

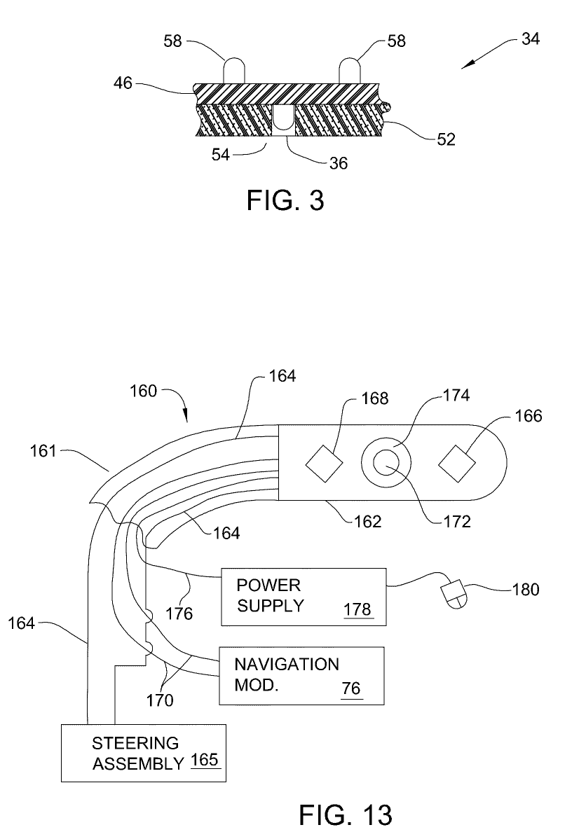

As seen in FIG. 3, in one version of the invention, a layer of foam 52 is also disposed over the distal side of substrate 46. Foam layer 52 is formed with a number of openings 54 (one shown). The individual LEEs 36 are disposed in openings 54.

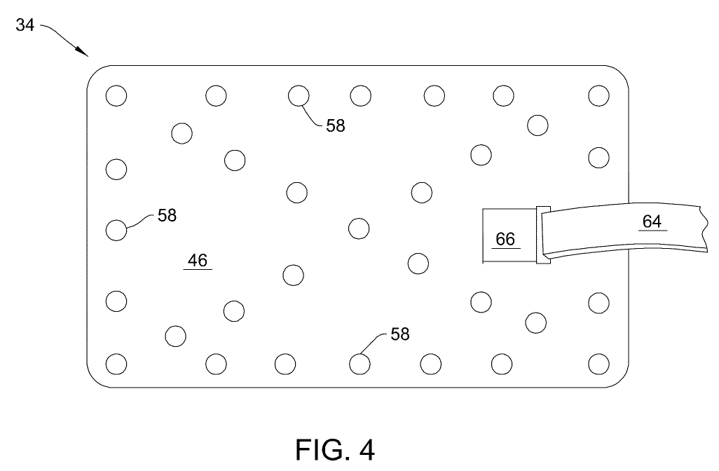

A second set of light emitting elements, LEDs 58, are attached to the proximal surface of substrate 46 as seen in FIG. 4. LEDs 58 emit light at a wavelength detectable by a localizer 60 that is part of navigation unit 38. In the illustrated version of the invention, LEDs 58 are arranged in lines located immediately inward of the outer perimeter of the substrate 46. Also two rows of LEDs 58 extend diagonally, from opposed corner to opposed corner, across the substrate. The two diagonal rows of LEDs 58 cross in the center of the substrate. Not shown are the conductors and load resistors that are connected to the LEDs 58. Also not shown in FIG. 4 are the conductors connected to the vias associated with distal side conductors 50.

A cable 64, shown as a ribbon cable, is also attached to the proximal surface of the light blanket substrate 46. Cable 64 includes individual conductors (not shown) that supply power to the LEEs 36 and LEDs 58. Not illustrated is the power supply to which the conductors are attached. It is understood that the exact structure of the power supply is not part of this invention. Cable 64 also includes signal conductors (not shown). The signal conductors supply instructional data signals generated by processor 40 to regulate the actuation of the individual LEEs 36 and LEDs 58.

A drive circuit 66, shown as a single integrated chip, is mounted to the proximal surface of substrate 46. Drive circuit 66 receives both the power and instruction signals received over cable 64. Based on the instruction signals, drive circuit 66 selectively connects each LEE 36 and LED 58 to the power supply so as to cause the actuation of these light emitting components.

Drive circuit 66 can be an application specific integrated circuit or a programmable logic gate array. In one version of the invention, drive circuit 66 includes a decoder and a set of power FETs. There is one power FET for each LEE 36 and LED 58. Alternatively, some LEEs 36 output variable amounts of light. In these versions of the invention, drive circuit 60 includes bipolar transistors for regulating the voltage or current applied to LEEs 36.

Processor 40 outputs over cable 64 to light blanket 34 instructions for regulating actuation of LEEs 36 and LEDs 58. In one version of the invention, these instructions comprise a stream of addresses. Each address corresponds to a specific LEE 36 or LED 58. A decoder, part of drive circuit 66, upon receipt of an address, momentarily turns on the FET associated with the specific LEE 36 or LED 58.

In a second version of the invention, drive circuit 66 contains a latch for each individually actuated LEE 36 and LED 58. Processor 40, as before, outputs a data stream containing the addresses of the individual LEEs 36 and LED 58. When the drive signal first receives the address for a specific LEE 36 or LED 58, the circuit toggles the latch so that the latch turns on the associated power FET. The next time that specific address is received by the decoder circuit, the circuit resets the latch. The resetting of the latch negates the output of the signal used to turn the power FET on.

As discussed above, some LEEs 36 are of the type wherein the amount of light they output can be selectively set. The instruction the processor outputs for this type of LEE 36 is in two parts. The first part comprises an address identifying the specific LEE 36. The second part is an operand. When the LEE 36 is to be turned on, the operand is a number indicating the magnitude of the current the drive should apply to the LEE. When the LEE 36 is to be turned off, the operand is a code indicating the drive circuit should deactivate the LEE.

Processor 40 is contained in a base unit 70. Casters 72 provide the base unit 70 with mobility. The navigation localizer 60 is mounted to the base unit 70 by a linkage that adjustably suspends the localizer above the base unit. Internal to the localizer 60 are cameras (not illustrated). The cameras are sensitive to the light emitted by light blanket LEDs 58.

Processor 40, in addition to being an overall part of system 30 of this invention, is also part of navigation unit 38. It should be appreciated that in, addition to an actual processing core, processor 40 includes a memory, represented as block 74 in FIG. 5. Memory 74 stores both instructions executed by the processing core of the processor 40 (processing core not illustrated) and the data processed and generated by the processing core. FIG. 5, in addition to illustrating memory 74 shows some of the main instruction modules executed by processor 40. To minimize drawing complexity, only some of the primary memory 74-module connections are shown to represent the reading of instructions and data from the memory and writing of instructions to the memory.

A navigation module 76 receives the signals captured by the localizer 60. Navigation module 76, it is understood, is part of navigation unit 38. (Prior to these signals being received by module 76, the signals may be digitized.) Based on these signals, navigation module 76 generates data indicating the free space locations of the LEDs 58. Here by “free space” it is meant that navigation module determines the locations of the LEDs 58 relative to the position of localizer 60. Based on the locations of the plural LEDs 58, navigation module also generates data indicating the position and orientation of the sections of the light blanket 34 between the LEDs 58. Localizer 60 and navigation module 76 may be similar to those found in the Applicants' Assignee's STRYKER® Navigation System

Commands to system 30, including navigation unit 38 and processor 40, are entered through a keyboard and mouse 80, also mounted to base 70. Information generated by system 30 and images generated by navigation unit 38 are presented on a display 82 also attached to base 70.

One type of procedure during which system 30 of this invention may be employed is a tissue reduction procedure. In this type of procedure, the photosensitive agent is actuated in order to cause the rapid die off, reduction, of a specific type of tissue. The tissue forming a tumor is one such type of tissue that is reduced using this procedure of this invention. Fat is a second type of tissue that is reduced using system 30.

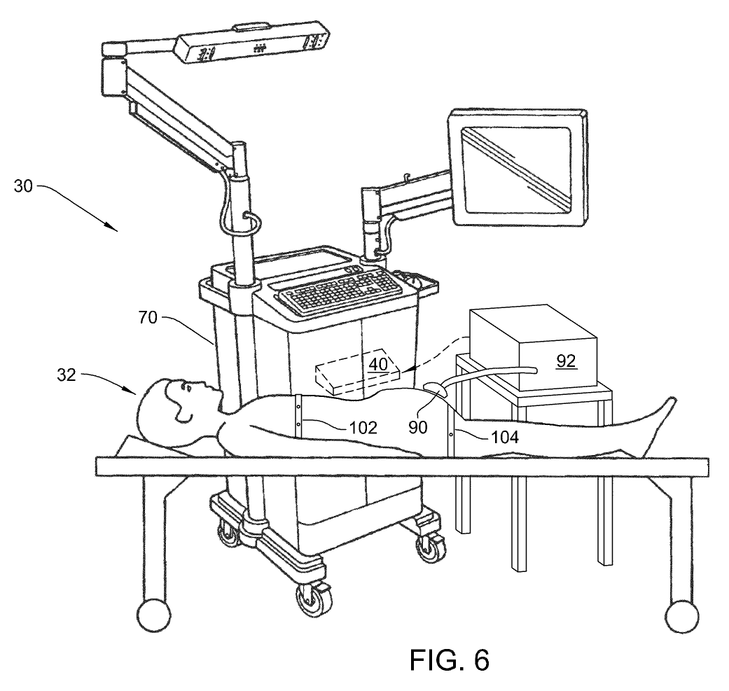

The procedure starts with the mapping of the body region containing the tissue that is to be reduced. In FIG. 6, the mapping of the tissue is shown as being performed by an ultrasonic imaging unit. The ultrasonic imaging unit includes a probe 90 and connected to a console 92 also part of the ultrasonic imaging unit. The probe 90 includes an array of transducers, not illustrated and not part of this invention, capable of emitting and sensitive to sonic waves, signals emitted generally between 1 and 13 MHz. The transducers are further capable of generating electrical signals upon return of an echo of the waves from the tissue under examination.

The ultrasonic imaging console 92 controls the actuation of the probe transducers. Console 92 also processes the signals output by the transducer upon their sensing of the returned echo. Based on the frequencies at which the transducers emit signals, the time it takes for the echo to be returned and the strength of the echo waves, console 92 generates data indicating the types of tissue located below the probe 90.

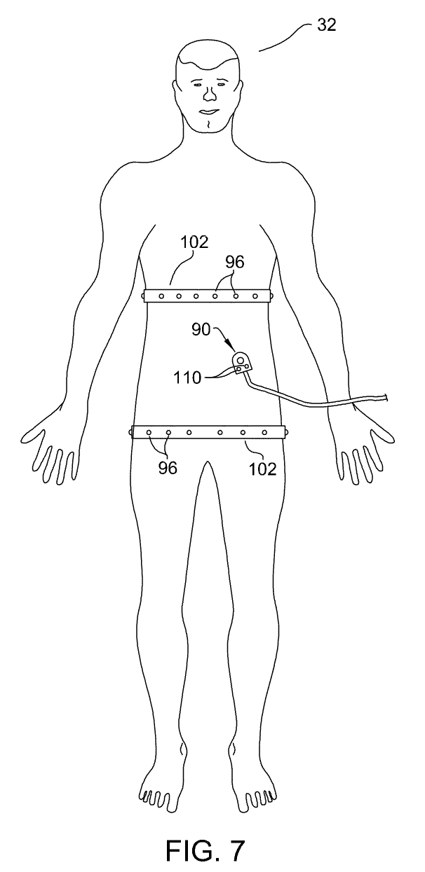

As seen in FIG. 7, as part of the mapping process a set of fiducial markers 96 are fitted over the patient 32. The fiducial markers 96 are devices the location of which can be tracked by the navigation unit 38. In the disclosed version of the invention, the fiducial markers are LEDs. These LEDs emit light that can be sensed by the cameras internal to the localizer. The fiducials are mounted to belts 102 and 104 that are worn by the patient during the mapping process and the part of the procedure in which the agent is activated. More, particularly belts 102 and 104 are worn over portions of the patient such that during minor movements of the patient the positions of the belts and therefore, the fiducials, are unlikely to shift. In FIG. 7 a first belt 102 is shown strapped to the chest of the patient immediately above the sternum, below the breast. Belt 104 is shown strapped to the patient immediately above the groin. To ensure that the navigation unit 38 is able to accurately generate a map of the outer surface of the body of the patient 32, belts 102 and 104 collectively have at least six (6) fiducials 96 the positions of which can be detected by the localizer. Further, belt 102 should be constructed so that there are opposed markers adjacent the opposed distals of the auxiliaries, (below the bases of the arm pits.)

Also, as seen in FIG. 7, disposed on top of ultrasonic probe 90 are LEDs 110. The light emitted by LEDs 110, like the light emitted by fiducials 96, is detectable by localizer 60. This allows the localizer to determine the position and orientation in free space of probe 90. The LEDs 110 fitted to probe 90 can collectively be considered the probe “tracker.”

Thus, during the mapping process, navigation module 76 receives data indicating the position of the patient fiducials 96, from these markers, the navigation module generates a dynamic reference map of the outer surface of the body of the patient 32. This map is referred to as “dynamic” because the patient may undergo movement while his/her position is being tracked.

Simultaneously with the tracking of the location of the patient, navigation unit 38 tracks the location of ultrasonic probe 90. Thus, during the mapping process, mapping module simultaneously receives data indicating the characteristics of the tissue below the probe 90 and the position of the probe on the patient 32. These data are presented to a tissue mapper 112 another set of instructions executed by processor 40. In FIG. 5 the data regarding the characteristics of the tissue are shown coming from an ultrasonic signal processor 92. This is the sub-circuit internal to the ultrasonic imaging console that generates the data defining the type of tissue present at a specific point within the body. Based on these two data streams, tissue mapper 112 generates a map of characteristics of the subcutaneous tissue of the patient. This map is stored in memory 74.

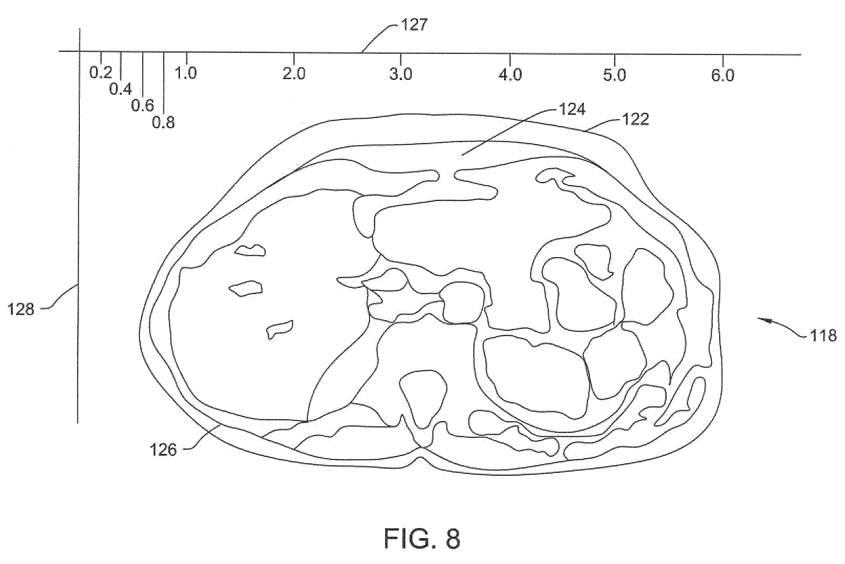

Once the tissue map is generated, it is available for viewing by the doctor. A utility module, not illustrated, retrieves the map from memory 74 and uses the map to generate image-forming signals that are applied to the display 82. FIG. 8 illustrates one such image 118 which is a slice view through the patient 32. Here, below the skin 122 there is a layer of transcutaneous fat 124. The organs of the thoracic cavity 126 are located below the fat 124. Associated with each point of tissue internal to the patient is a reference point. In FIG. 8 these reference points can be considered to be taken relative to the common origin point of an abscissa axis 127 and an ordinate axis 128. Index points are shown extending from axis 127. In practice it is understood that the addressable tissue locations are spaced very close together.

The medical practitioner reviews the map to determine the locations at which the tissue is to be reduced. For example, in a procedure to remove fat, tissue present at certain locations may be the fat tissue to be subjected to removal. In a cosmetic procedure, the practitioner may review the images with the patient. Jointly, the practitioner and the patient may decide which tissue is to be subjected to reduction.

Once a decision has been made to reduce specific tissue, that tissue is marked. This process is performed by a target mapper 130, a software module also run on processor 40. One input to target mapper 130 are the data defining the tissue map, retrieved from memory 74. A second input into the target mapper 130 is the doctor-generated data indicating which tissue is targeted for treatment. The doctor can use any convenient process to identify the tissue selected for treatment. This process may be performed using a mouse on an image of the body section. Alternatively, the tissue images are presented on a touch screen. An appropriate complementary marking pen is then used to define the borders of the tissue that is to be marked.

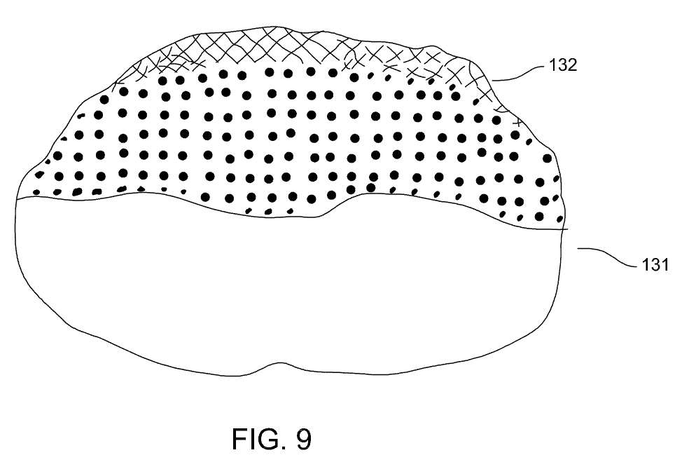

As a result of the doctor indicating which tissue is selected for treatment, target mapper 130 generates a map of the body section on which the locations of the tissue to be flagged are marked. An image 131 of this map is presented on FIG. 9. Here irregularly curved line 132 represents the skin of the patient. A location wherein the tissue is to be reduced is marked by “×”s. Tissue that is not to be subjected to the reduction therapy are marked with “·”s. The map generated by the target mapper 130 is stored in memory 74.

After the tissue flagging process is completed, the actual reduction process is initiated. This process begins with the introduction of an appropriate photosensitive therapeutic agent into the patient 32. One agent that can be used in this process is taporfin sodium, (LS11). If this is the employed agent, light emitting elements 36 should emit light between 650 and 680 nm and, more ideally, at 664 nm. The actual process by which the agent is introduced into the portion of the body in which the procedure is to be performed may vary with the nature of the agent, the location at which the procedure is to be performed and/or the type of tissue on which the procedure is to be performed. For example some agents are introduced intravenously into the body and are allowed to diffuse throughout the body. Other agents are injected into the body at the location at which the procedure is to be performed.

When the system and method of this invention is used to activate agent so introduced, the invention minimizes the activation of the agent that diffuses into nearby normal tissue.

Once the patient's tissue has been given time to absorb the photosensitive agent, the agent is selectively actuated using the assembly of FIG. 1. Specifically, the patient lies on the table while the fiducial markers (LEDs 96) remain affixed to his/her body.

Light blanket 34 is placed over the portion of the patient containing the tissue to be reduced. Owing to the flexible nature of substrate 46 and foam 52, the light blanket 34 generally conforms to the contours of the patient. The foam 52 prevents sections of the substrate 62 from bunching together.

Proximally directed light blanket LEDs 58 are then actuated. Based on the LED light received by localizer 60, navigation module 76 generates a free space map of the location of the light blanket 34. More particularly, based on the above data, navigation module 76 generates data indicating over which portion of the patient 32 each LEE 36 is disposed.

Then, for each LEE 36, an LEE driver 136, also executed by processor 40, determines whether or not the LEE should be actuated and the overall quantity of light the LEE should emit. This determination is the result of a three-part process. In the first part of the process, LEE driver 136 generates a map of the position of each LEE 36 relative to the underlying targeted and non-targeted tissue. One input LEE driver 136 are the data from the navigation unit 60 that indicates over which portion of the patient each LEE 36 is disposed. A second input into the LEE driver 136 are the retrieved from memory 74 map indicated if the tissue present at a particular location is targeted or non-targeted.

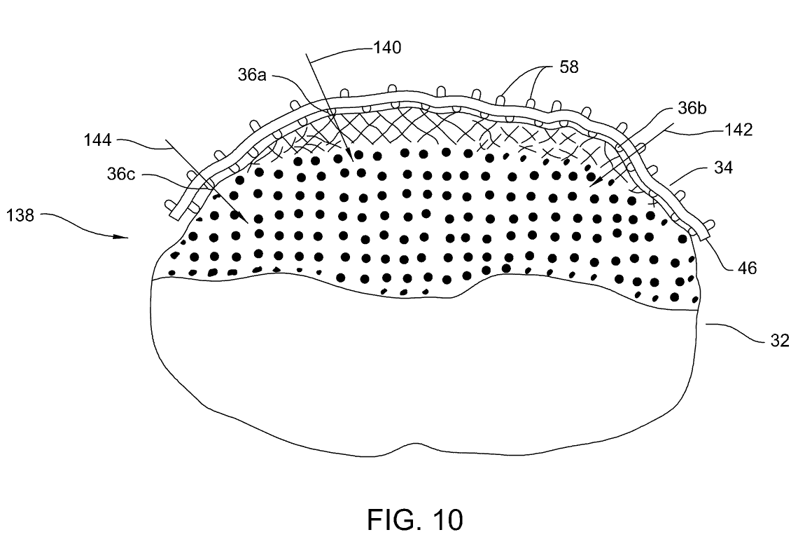

Diagrammatically, in this part of the process, LEE driver 136 effectively generates the image 138 of FIG. 10. In this Figure, the presence of the blanket substrate 46 and blanket LED 58 are also shown. To simplify the image, the foam layer is not shown.

In the second part of this process, LEE driver 136, maps for each LEE 36 the path of the light output by the element through the patient. Each of these paths can be considered a vector that originates from the associated LEE 36. The origin and direction of each vector is based on the position and orientation of each LEE 36. In FIG. 10 arrows 140, 142 and 144 represent the paths of light emitted from three separate LEEs 36a, 36b, and 36c, respectively, through the underlying targeted and non-targeted tissue.

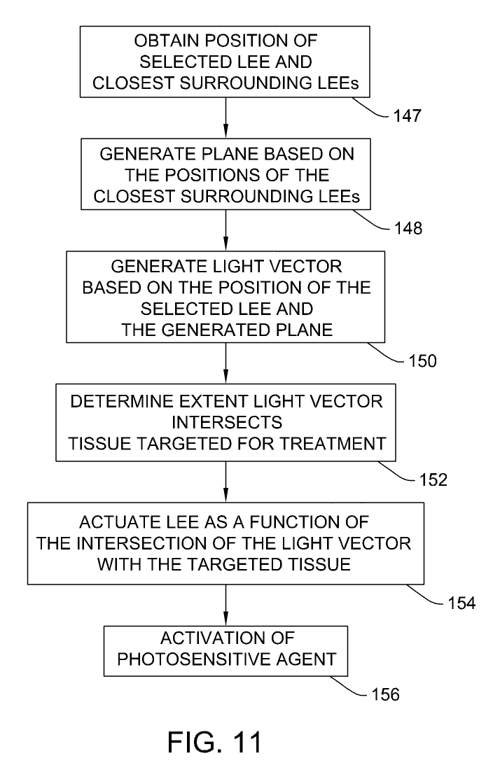

One process by which each light vector is mapped is now explained by reference to FIG. 11. Initially, in a step 147, LEE driver 136 obtains the position of the LEE 36 for which it is going to generate the associated light vector and the closest LEEs 36 surrounding the selected LEE. Ideally the “closest surrounding” LEEs 36 are the four closest LEEs that define a quadrilateral the spatial projection of which contains the selected LEE. If the selected LEE 36 is on the edge or corner of the light blanket 34, the “closest surrounding” LEEs may be simply the three closest LEEs.

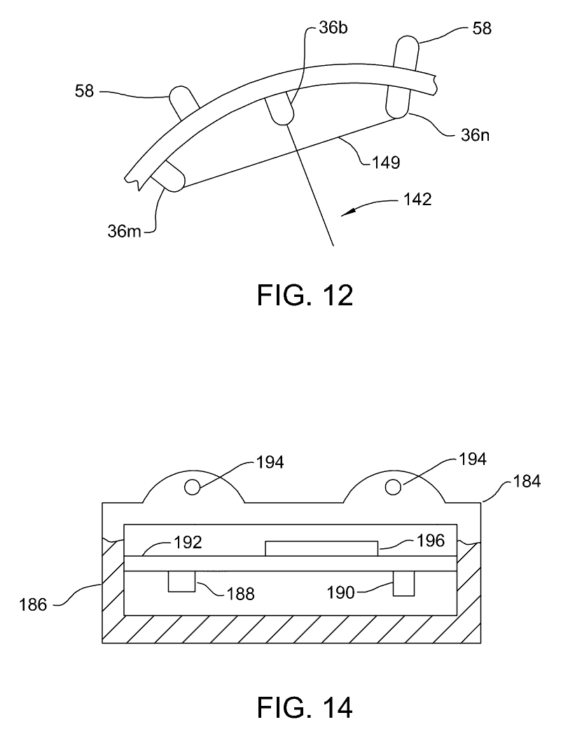

Then, in a step 148, using a best fit algorithm, LEE driver 136 defines a plane using as the points of reference the locations of the closest surrounding LEEs 36. In a step 150, the light vector is generated. More particularly, the LEE driver 136 calculates the vector being one that has its origin the location of the selected LEE 36. The second point of reference for this vector is the point where the vector intersects the previously defined plane along a line that is perpendicular to the plane. FIG. 12 is a representation of the plot of this vector in two dimensions. In this Figure, line 149 between LEEs 36 represents the plane that surrounds LEE 36b. Here, line 149 extends between LEE 36m and LEE 36n. In a three dimensional model line 149 lies in the plan defined by the LEEs that surround the LEE being modeled.

In the third part of the process by which the control of each LEE 36 is set, LEE driver 136 then determines the extent to which the light emitted by a particular element will intersect tissue targeted for treatment. In FIG. 11 this is represented by step 152. Based on this determination, LEE driver 136 determines if a particular LEE 36 should be actuated and, if it is to be actuated, the amount of light the element should output. For example, the light generated by LEE 36a that travels along vector 140 intersects a relatively thick region of tissue selected for treatment. Based on this determination, LEE driver 136 recognizes that it is appropriate to have LEE 36a, emit a relatively large quantity of light. The light generated by LEE 36b, the light emitted along vector 142, will intersect a thinner section of tissue targeted for treatment. Based on this intermediate analysis, LEE driver 136 module recognizes that, while LEE 36b should be actuated, the amount of light it should emit should be less than that emitted by LEE 36a.

As a result of the plotting of light path vectors over the targeted tissue map, the LEE driver 136 recognizes that the light emitted by LEE 36c, the light traveling along vector 144, will not intersect any tissue flagged for treatment. Based on this analysis, LEE driver 136 recognizes that it is not appropriate to actuate LEE 36c.

As a result of the determinations of the extent to which each LEE 36 should be actuated, LEE driver 136 causes processor 40 to output instruction signals to the light blanket, step 154. The instruction signals for each LEE 36a, 36b, 36c . . . cause the LEE to be actuated as appropriate. For some light blankets, the actuation signals cause each LEE to be actuated for a specific amount of time. The exact amount of time is directly proportional to the quantity of light LEE driver module determined the element should emit. For some light blankets, the actuation signals are used to regulate both the actuation of the individual elements as well as the intensity of the light each emits. If the LEEs 36 are diodes or lasers, light intensity may be regulated by sending commands to drive circuit 60 that regulates the current applied to each element to be actuated.

As a consequence of the selected application of light, photonic energy, into the patient, the activation of the photosensitive agent is precisely regulated, step 156. The agent absorbed in a large mass of tissue selected for treatment is exposed to an appreciable quantity of light to result in a relatively full activation of the agent. The agent absorbed by a smaller mass of tissue selected for treatment is exposed to a lesser quantity of light. This ensures that at least some of the agent in the targeted tissue is actuated while minimizing the unneeded actuation of the agent in nearby normal tissue. Similarly, the extent to which light is introduced into large mass of normal tissue in which the agent may be absorbed is substantially eliminated.

Thus, the system and method of this invention, maximizes the activation of the agent in tissue targeted for treatment while simultaneously reducing the extent to which light activates the agent in nearby normal tissue. System 30 therefore makes it possible for the patient to obtain the relatively complete benefit of the activation of the agent where needed while minimizing its potential undesirable activation in normal tissue.

FIG. 13 illustrates the distal end of a catheter 160 constructed in accordance with this invention. Catheter 160 includes a flexible elongated body 161 adapted to be inserted into a patient. Some versions of catheter are adapted to be insertable into a vein. Still other catheters 160 of this invention are adapted to be threaded between and/or into internal organs.

Catheter 160 has a distal end tip 162 formed of rigid plastic. The catheter 160 is steerable. Thus, as is known in the art, two or more guide wires 164 (two shown) extend through the catheter and are attached to the proximal end of tip 162. The selective tensioning and slacking of guide wires 164 moves the catheter tip 162 to control the direction of advance of the catheter. In FIG. 13, guide wires 164 and 164 are shown connected to a steering assembly 165. The steering assembly 165, in response to commands entered by the doctor, is the sub-assembly that actually tensions and slacks guide wires 164.

Disposed in catheter tip 162 are two longitudinally spaced apart magnetic field transducers 166 and 168. Each transducer 166 and 168 includes a set of individual sensors (not illustrated). The sensors sense the magnetic fields present in three mutually orthogonal axes. In one version of the invention, each transducer consists of a square block. Three mutually orthogonal coils (not illustrated) are wrapped around the outer surfaces of the block. The coils function as the three sensors.

Shown extending proximally from catheter tip 162 are two conductors 170. Conductors 170 are the conductors over which the signals generated by the sensors integral with transducers 166 and 168 are output. Conductors 170 are shown connected to the navigation module 76. As explained below, based on the signals of magnetic strength received by the transducer sensors, the navigation module generates data indicating the position of the catheter tip when disposed in the patient.

Also mounted to catheter tip 162 is a light emitting element 172. Light emitting element 172, (an LED or a laser diode) emits light at a wavelength that can trigger the activation of agent absorbed by tissue. Light emitting element 168 is disposed between transducers 166 and 166. A shield 174 formed from impermeable material surrounds light emitting element 172. Shield 174 thus prevents electromagnetic waves emitted by light emitting element 172 from being sensed by either of the transducers 166 and 168.

A conductor 176 extends through the catheter body to the catheter tip 162. Conductor 176 is the member over which signals are applied to the light emitting element 172 to actuate the element. The conductor 176 is shown connected to a power supply 178. Not shown is the ground connection that may be connected from the light emitting element 172 to the power supply. The surgeon actuates a trigger 180, shown as a mouse, to cause the power supply to actuate, turn on, the light emitting element 172.

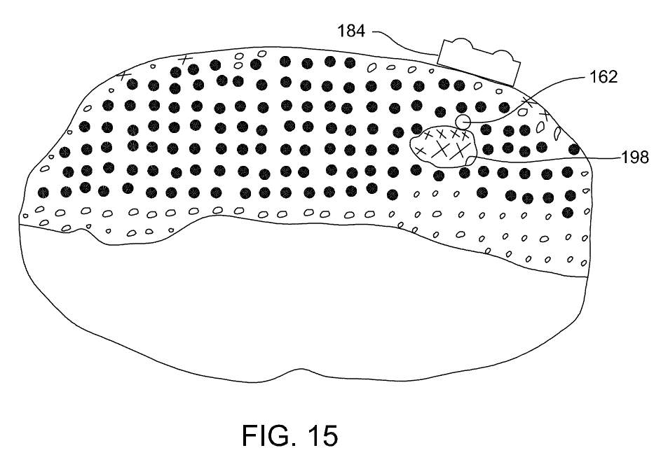

Navigation unit 38 monitors the position and orientation of catheter 160 using a tracker 184 now described by reference to FIGS. 14 and 15. Tracker 184 includes a shell 186 that in some preferred versions of the invention has a generally rectangular footprint. The surface area occupied by shell 186 is usually less than 230 cm², preferably less than 130 cm² and more preferably 80 cm² or less. Internal to tracker shell are two transmitter assemblies 188 and 190. Each transmitter assembly 188 and 190 is capable of transmitting electromagnetic signals along three mutual orthogonal axes. In most versions of the invention, each transmitter assembly 188 and 190 contains three coils that are mutually orthogonal to each other.

Transmitter assemblies 188 and 190 are mounted to a planar substrate 192 disposed in the shell 186. The transmitter assemblies 188 and 190 are mounted to the distal facing side of substrate 192 so as to be directed towards the patient 32.

Each tracker assembly includes plural LEDs 194. The LEDs 194 are mounted to semi-cylindrical bosses that extend above the top planar surface of shell 186. LEDs 194 emit light outwardly and at a wavelength that is detectable by navigation unit localizer. Also disposed in tracker shell 186 and mounted to substrate 192 is a control module 196 shown as a single rectangular block. Control module 196 contains a set of adjustable current sources, (not illustrated). Each current source regulates the current applied to a separate one of the coils of a specific transmitter assembly 188 or 190. Control module 196 also contains components that regulate the actuation of LEDs 194. A more detailed understanding of the construction of tracker 184 is found in the Applicants' Assignee's U.S. patent application Ser. No. 11/333,558, HYBRID NAVIGATION SYSTEM FOR TRACKING THE POSITION OF BODY TISSUE, filed 17 Jan. 2006, now U.S. Patent Pub. No. US 2007/0225595 A1, the contents of which are incorporated herein by reference.

Catheter 160 is used to activate a photosensitive agent absorbed in tissue spaced below the outer layer of the body cavity. When it is necessary to activate tissue so located, catheter 160 is threaded, inserted, moved or otherwise advanced towards the tissue. To keep track of the position and orientation of the distal end of catheter 160, tracker 184 continually actuates transmitter assemblies 188 and 190. The electromagnetic waves emitted by these transmitters are sensed by catheter transducers 166 and 168. The signals representative of sensed magnetic field strength are transmitted through the catheter body 161 back to navigation module 76. Based on these signals, navigation module generates data indicating the position and orientation of the transducers 166 and 168 relative to the tracker transmitter assemblies 188 and 190. Algorithms such as those provided in U.S. Pat. No. 4,287,809, Helmut-Mounted Sighting System, issued 8 Sep. 1981, U.S. Pat. No. 4,314,251, Remote Object Position And Orientation Locator, issued 2 Feb. 1982 and U.S. Pat. No. 4,945,305, Device For Quantitatively Measuring The Relative Position And Orientation Of Two Bodies In The Presence Of Metals Utilizing Direct Current Magnetic Fields, issued 31 Jul. 1990 are employed to, based on the magnetic field measurements, determine the position and orientation of the catheter transducers 166 and 168. Each of the above-cited documents is incorporated herein by reference.

At manufacture, the position and orientation of the catheter light emitting element 172 relative to transducers 166 and 168 is determined. Therefore, by extension, once navigation module 76 determines the position and orientation of transducers 166 and 168 to the tracker transmitters 188 and 190, the module determines the position and orientation of the light emitting element 172 to the tracker transmitters.

Integrated with the determination of the position of the catheter light emitting element 172 relative to the tracker transmitter assemblies 188 and 190, the navigation localizer 60 receives the light waves emitted by the tracker LEDs 194. Based on the measurement of these light waves, navigation module 76 determines the free space position and orientation of the tracker 184.

Navigation module 76 is thus able to determine the free space position and orientation of the catheter light emitting element 1172 according to the following formulas:

\vec{x}_{L→C}=\vec{x}_{L→T}+R_{L÷T}·\vec{x}_{T→C} (1)

and

R_{L→C}=R_{L→T}·R_{T→C} (2)

Here, \vec{x}_{L→T} is the vector from localizer 60 to tracker 184. Vector \vec{x}_{T→C} is the vector from the tracker 184 to the catheter light emitting element 172. Vector \vec{x}_{L→C} is, therefore, the vector from the localizer to the catheter light emitting the localizer to the catheter light emitting element. Matrix R_{L→T} is the rotational matrix from the x-, y- and z- axes of the localizer to the corresponding axes of the tracker. Matrix R_{T→C} is the rotational matrix from the x-, y- and z-axes of the tracker 184 to the corresponding axes of the catheter light emitting element 172. Matrix R_{L→C} is, therefore the rotational matrix from the axes of the localizer 60 to the corresponding axes of the catheter light emitting element.

Therefore as a result of this processing, navigation module 76 determines the position and orientation of the catheter light emitting element 172 in free space. Using the previously discussed process, navigation module 76 tracks the position and orientation of the patient's body. The navigation module 76, using the foregoing data as well as the map of the tissue internal to the patient, maps the position and orientation of the catheter light emitting element 172 relative to the tissue targeted for treatment, mass 198 in FIG. 15.

When the light emitting element is both adjacent the targeted tissue and directed towards the tissue, the doctor, by actuating trigger 180, causes power supply 178 to energize the catheter light emitting element 172. Alternatively, the actuation can be automatic. In this version of the invention, actuation is based on a determination that the light emitting element 172 is both orientated towards and within a specific distance of the tissue targeted for treatment.

The above-described version of the system and method of this invention, is used to actuate a photosensitive agent that may be tissue located so deep within the body that light emitted from outside is absorbed by intermediate tissue. This version of the invention can also be employed when the tissue between skin layer and the target tissue may be normal tissue that could absorb an appreciable quantity of the agent. Therefore, use of this system and method of the invention prevents the unwanted triggering of this intermediate located agent.

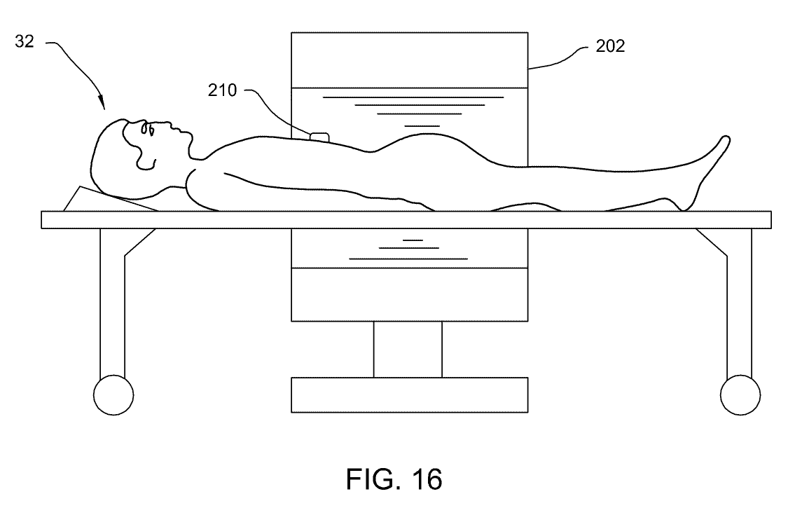

As shown in FIG. 16 an alternative diagnostic unit may be used to map the tissue prior to the treatment process. Here the diagnostic unit is a magnetic resonance imager (MRI) 202. The MRI 202, based on difference in spin of the atomic nuclei in the patient's tissue, generates map data of the different tissue internal to the patient.

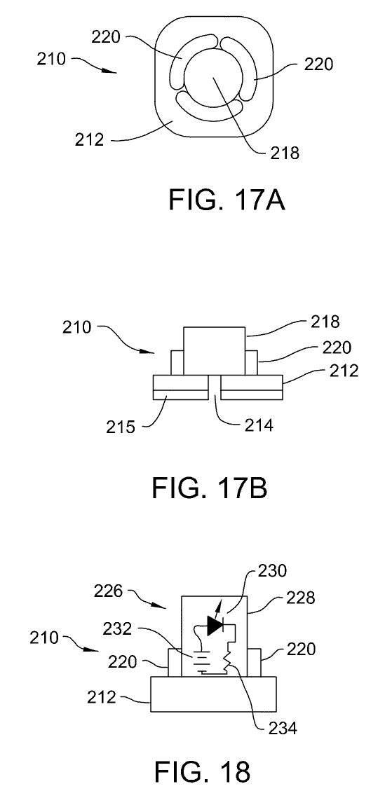

Also seen in FIG. 16 is one fiducial marker 210 used to generate a reference frame between the MRI generated map of the patient and the position of the patient as determined by the navigation unit 38. As seen in FIGS. 17A and 17B, each fiducial marker 210 has a plastic, planar rectangular base 212. Marker base 212 is formed so as to define in the center a through hole 214 the purpose of which will become apparent below. An adhesive layer 215 is disposed over the distal, patient facing, side of the marker base 212. The adhesive is of the type that can be used to temporarily secure the marker 106 to the patient.

A magnetic marker 218 is removably mounted to the top, proximal face of marker base 212. Marker 218 is formed of material that generates a distinct image when subjected to magnetic resonance imaging. One such material is a gadolinium liquid marketed by Beekley Corporation of Bristol, Conn. under the trademark RADIANCE. This liquid is contained in a capsule that forms the body of the marker. The magnetic marker 218 is removably held to the fiducial marker 210 by tabs 220 that are integrally formed with and project upwardly from marker base 212. Tabs 220 are arcuately shaped and centered around base through hole 214. When the magnetic marker 218 is fitted to base 212, the marker 118 thus covers through hole 214.

Three or more fiducials 210 are affixed to the patient in order to provide a reference in three-dimensional space for the coordinates of the body map. The fiducials 210 are placed at locations around the portion of the patient on which the procedure is to be performed that remain essentially constant relative to the procedure location. For example, if the procedure is to be performed on the tissue within the abdomen, one fiducial 210 is placed on the skin above the base of the sternum, markers are placed on the opposed distal of the auxiliary, (at the base of the arm pit,) markers are placed on the opposed sides of the waist and a marker is placed at the groin. Thus, six (6) markers are placed on the patient.

At the start of the procedure in which the photosensitive agent is to be activated, magnetic markers 218 are removed from the fiducial marker bases 212. A pointer (not illustrated,) an instrument tracked by navigation unit 38, is then touched to the tissue accessible through the now-exposed hole 214 in each fiducial 210. The tissue mapper 112 can then be provided with the data from the MRI-generated tissue map and the data from navigation unit 38 regarding the location of fiducial 210. Based on these two sets of data, tissue mapper 112 superimposes the MRI generated tissue map onto the free space map of the patient.

In the foregoing arrangement, it may still be necessary to, during the agent activation procedure, have the patient wear fiducial markers 76. This will allow movement of the patient 32 and, by extension, the internal tissue map, to be tracked during the process of positioning the agent-activating LEEs.

As an alternative to placement of the fiducial markers on the patient 32, once each magnetic marker 218 is removed from each fiducial 210, a navigation marker 226, seen in FIG. 18, may be attached. Each navigation marker 226 includes a housing 228. The housing 228 is designed to be snuggly fitted to fiducial 210 by tabs 220. Internal to housing 228 is an LED 230 that emits light at a wavelength detectable by localizer 60. Also internal to the housing 228 is a battery 232 for energizing the LED 230. A load resistor 234 is shown connected in series with LED 230 and battery 232. Not shown is a control circuit internal to housing that regulates the pattern in which LED 230 is actuated. This may be necessary so each LED 230 emits light in a unique pattern. This is desirable so the navigation module 76 is able to distinguish the light from the individual markers on the patient, the light blanket and/or tracker.

When navigation marker 226 is fitted to fiducial 210, navigation unit 38 monitors the light emitted by the LED 230. In this way, navigation unit 38 is able to provide data regarding the dynamic position of the patient in free space. From these data and the MRI data, tissue mapper 112 generates an image of the real time location of the tissue internal to the patient targeted for treatment.

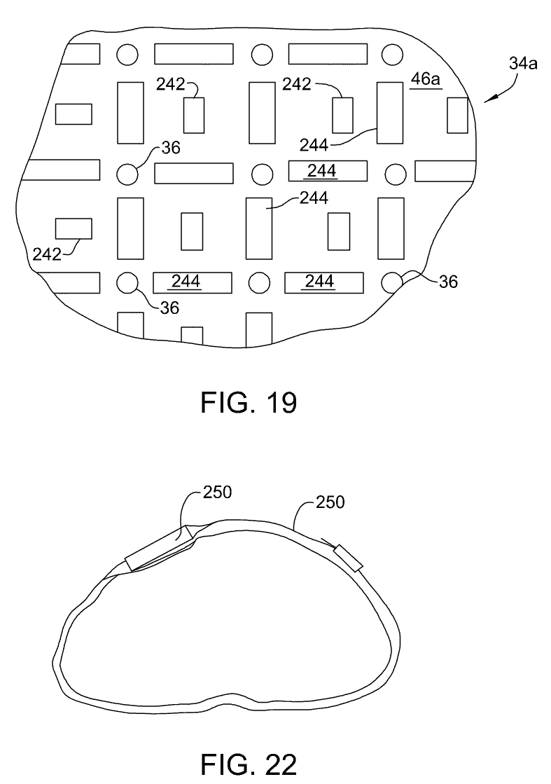

FIG. 19 is the distal facing side of an alternative light blanket 34a of this invention. Light blanket 34a includes a substrate 46a to which LEEs 36 are mounted. Also attached to substrate 46a are plural ultrasonic transducer units 242. The transducer unit 242 is connected to a common ultrasonic signal processor 92 (FIG. 5). A number of Peltier cooler plates 244 are also attached to substrate 46a. A Peltier cooling plate includes a set of thermocouples. When a voltage is applied to the thermocouple junctions, heat flows between the junctions. Thus, one of the sides of the cooling plate is side from which heat flows. This side is the cooling surface of the cooling plate.

Blanket 34a is used to both perform the diagnostic step of the method of this invention and emit the light needed to perform the therapeutic process. Specifically, at the start of the process, blanket 34a is fitted to the patient so as to remain fixed relative to the patient. The individual ultrasonic transducers 242 are then actuated and the signals emitted therefrom are applied to ultrasonic signal processor 92. Based on the signals received from the transducers 242, the tissue map of the patient is developed.

Then, using the above described procedures, the tissue to be treated is identified. Using the blanket 34a of this version of the invention, prior to the introduction of the therapeutic agent into the patient, Peltier cooling plates 244 are actuated. This results in the cooling of the patient's skin. Once the patient's skin is so cooled, the therapeutic agent is introduced into the patient.

The cooling of the patient's skin with the blanket 34a contracts the blood vessels leading to the skin and the capillaries internal to the skin. The contraction of these blood vessels and capillaries reduces the extent to which the therapeutic agent is able to diffuse into the skin.

Then, using the previously described processes, the individual LEEs 36a are actuated.

Light blanket 34a does more than simply provide a set of LEEs the locations of which are easily identifiable and that are individually actuatable. Light blanker 34a, also includes a set of diagnostic transducers useful for precisely identifying the tissue that is to be treated. A light blanket 34a remains fixed to the patient during initial diagnostic phase of the process and stays fixed through the treatment phase. Therefore the possibility of there can be shifting in the positions of the LEEs 36 relative to the mapped tissue is substantially eliminated.

Still another benefit of blanket 34a is that it does more than serve as a combined diagnostic and treatment tool. The Peltier cooling plates 244 reduce unwanted diffusion of the therapeutic agent into the skin and tissue immediately underlying the skin. This reduces the extent to which large quantities of agent in this tissue is needlessly actuated.

Further, as a function of the tissue targeting process, the activation of the Peltier cooling elements can be individually set. Thus it may be desirable to only nominally activate the Peltier cooling elements 244 located over tissue that is to be treated. This ensures the relatively large absorption of agent in tissue to be treatment. Similarly, the Peltier cooling elements 244 located over normal tissue may be actuated to maximize cooling. This step minimizes the absorption of the agent in this tissue. Thus, by both cooling the normal tissue to reduce its absorption of the therapeutic agent and minimizing the activation of agent that is absorbed in this tissue, this invention reduces extent to which the agent is, normal tissue adjacent tissue targeted for treatment, activated.

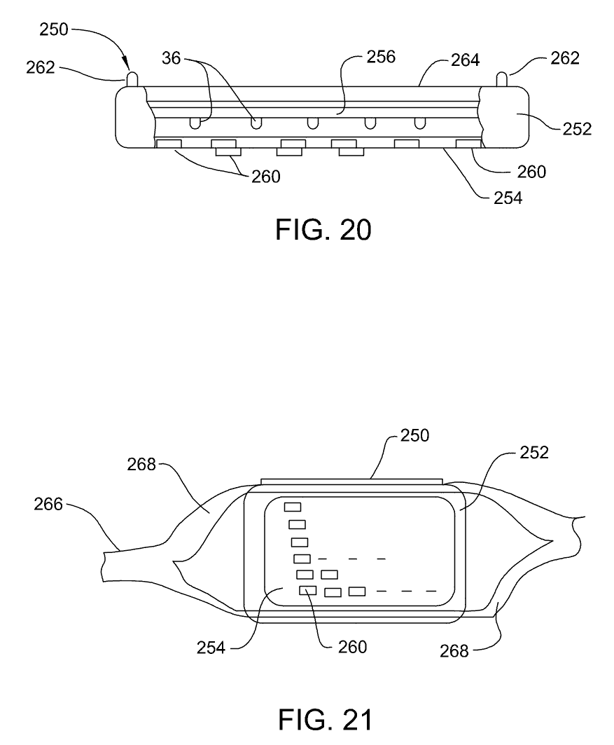

FIG. 20 illustrates a light plate 250, an alternative activation unit of this invention. Light plate 250 includes a rigid frame 252. Attached the frame is a distal facing plate 254 that is transparent to the light needed to activate the photosensitive agent. Disposed inside frame 252 is a substrate 256. Mounted on the distally directed surface of substrate 256 are a number of LEEs 36. Mounted to plate 254 are a number of ultrasonic transducers 260. Transducers 260, like transducers 242, are connected to ultrasonic signal processor 92

A set of LEDs 262, the light of which can be sensed by localizer 60 are mounted to the proximally directed face of frame 252. A cover 264 disposed over substrate 256 forms the proximally directed face plate of light frame 250.

Light frame 250 is secured over the portion of the patient containing the tissue to be treated by a strap 266 seen in FIGS. 21 and 22. Strap 266 is formed with a number of elastic bands 268 that hold the frame 250 in place and that are spaced from LEEs 36, transducers 260 and LEDs 262.

To selectively activate an agent using light frame 250, the frame, with the aid of strap 266, is pressed against the patient above the tissue to be activated. More particularly, the frame is positioned so that plate 254 presses against the skin of the patient. The light emitted by LEDs 262 is used to plot the position of the light frame 250 relative to fiducial markers on the patient. Transducers 260 are used to map the tissue below the light frame. LEEs 36 are used to selectively activate the agent absorbed in the underlying tissue.

Light frame 250 is constructed so that positions of the LEEs 36, transducers 260 and LEDs 262 are fixed relative to each other. This facilitates the relatively simple determination of which LEEs 36, relative to the targeted tissue, should be actuated.

Further, strap 266 presses the light frame 250 against the skin against which the frame is disposed. This pressure compresses the veins and capillaries through which blood is flowed to the skin. The compression of these capillaries and veins reduces the extent to which agent that is introduced into the blood stream is absorbed by this section of skin. Thus, when various LEEs in light frame 250 are actuated, the photonic energy emitted by these LEEs does not activate large amounts of skin-located agent.

The foregoing is directed to specific versions of this invention. It should be clear that this invention may have structures that vary from what has been described. Thus, alternative means may be used to: map the tissue internal to the patient 32 or track the location of the light emitting elements.

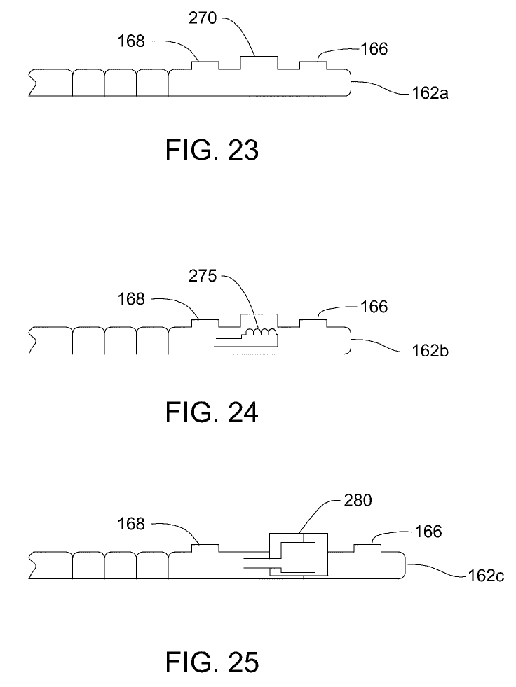

Also, this invention is not limited to systems that only trigger agents that are actuated by the application of photonic energy. In one an alternative version of the invention, the energy-emitting device may be a device that emits sonic or ultrasonic energy. A piezoelectric device, such as the piezoelectric transducer 270 shown in FIG. 23, may substitute for the LEE attached to catheter tip 162a. This version of the invention is used to trigger the activation of a pharmaceutical agent that is activated upon the application of mechanical vibrations. For example, there are pharmaceutical compounds that consist of compounds encased in small millimeter or less, and more often micrometer or less, shells. These shells, which are only a few molecules thick themselves are ruptured open upon being exposed to mechanical vibrations.

In these versions of the invention, the pharmaceutical compound is introduced into the body in the vicinity of the tissue where the encased agent will have the desired therapeutic effect. Using one of the above techniques the system only activates the sonic/ultrasonic transducers that will emit energy towards the tissue to be treated. This energy induces vibrations in the shells encapsulating the agent. The vibrations cause shell rupture and the release of the agent contained therein.

A benefit of using the system of this invention to so release the encapsulated agent is that only agent in the vicinity of the tissue to be treated is so released. Encapsulated agent spaced from the tissue remains in its shell. Over time, this encased agent is filtered and excreted from the body.

It should likewise be appreciated that ultrasonic transducers or other energy emitters may be attached to the distal face of a blanket or other support structure pressed against the outer surface of the patient. Navigation markers are disposed on the opposed proximal side of the support structure.

Alternatively, the system of this invention can include devices for selectively emitting either

For example, certain chemical reactions are known to occur more rapidly in the presence of a magnetic field. One such reaction is the creation of Heat Shock Protein Hsp70 (Dnak in E. coli). This protein is a chaperone protein. It is believed that this protein crowds an unfolded substrate, stabilizes it and prevent aggregation until the unfolded molecule folds properly. Once the unfolded molecule so folds, the Hsp70 proteins lose affinity to the molecule and diffuse. The increased concentration of Hsp70 protein reduces a cell's tendency towards, apoptosis, cell death. Hsp70, like other heat shock proteins, develop when a cell is subjected to stress, such as heating.

Thus, by using the system of this invention, a concentrated field of electromagnetic energy, the formation of Hsp70 proteins in a specific section of tissue or organ can be fostered. This can be useful to prevent damage to tissue that is prone to damage as a consequence as a result of a stroke, heart attack or other condition.

The transducer 275 attached to catheter tip 162b of FIG. 24, can therefore be one that selectively emits RF energy. This is useful when the agent is activated by the application of thermal energy. This is because a focused beam of RF energy will heat the tissue to which it is applied.

Alternatively, thermal energy could be applied to or withdrawn from a compound that functions as the carrier for the active agent. For example some compounds are known to form a semisolid gel when heated. In some uses of the system of this invention, a pharmaceutical agent is mixed into the gel forming compound and then introduced into the body in the vicinity of the tissue to which the agent is to be applied. The system of this invention is then used to heat the fraction of the blended compound adjacent the tissue that is to be subjected to therapy. In these versions of the invention, the catheter tip may have a transducer 275 heating element, or a RF coil.

When the navigation system determines that the transducer is adjacent the tissue to be subjected to therapy, the transducer is actuated to warm the adjacent environment. More particularly, what occurs as a result of this warming is the state change of the blended compound from a liquid to a semisolid get that coats the tissue. The active agent at the surface of the gel is thus applied to the tissue in order to cause the desired therapeutic effect. The remaining portion of the blended compound that is the liquid state is filtered and excreted from the body.

Alternatively, the system of this invention may include a transducer that does not actually “emit” energy but, instead functions as an energy “sink.” This version of the system is useful for fostering the desired chemical reactions or inhibiting undesirable reactions at a target location in the body in order to ensure the desired therapeutic result. Thus, in some instances the agent or tissue is cooled to foster the occurrence of chemical reactions that would not normally occur at the internal body temperature. Alternatively, the agent is cooled to prevent an undesirable chemical reaction. This later situation may be the case if the agent is cooled to prevent a reaction from occurring outside the tissue that would result in the reduction in concentration inside the tissue that can effect the desired therapy.

By way of example, FIG. 25 illustrates how a Peltier cooling assembly 285 may be mounted to a catheter tip 162c of this invention.

In either situation, using the steering assembly and the navigation system, catheter tip 162c is positioned adjacent the location at which the desired chemical reaction should occur (or the undesired reaction not take place.) When the catheter tip 162c is so positioned, the Peltier effect transducer or other heat sink device is actuated to cool the surrounding agent.

Thus, it should be understood that the system of this invention not only triggers a desired reaction by applying energy to a body site, it can do so by functioning as a sink that selectively draws energy away from the site.

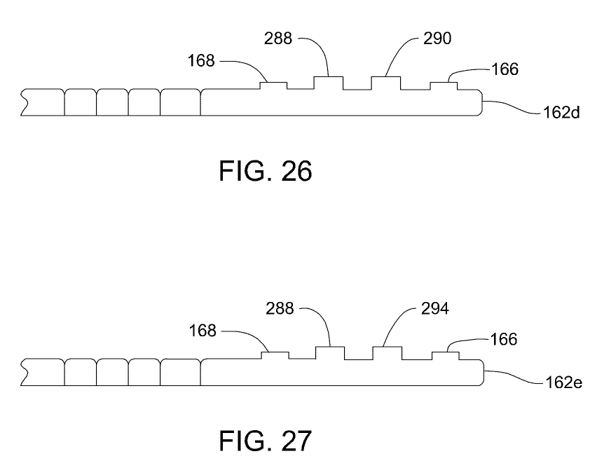

FIG. 26 illustrates an alternative catheter tip 162d of the system of this invention. Tip 162d includes the previously described magnetic field transducers 166 and 168, which can be considered navigation markers for the tip. An energy emitting (or sinking) component such as one of the above described is represented as 288. Component 288 can thus be one of the described devices that emits light, EM energy, thermal energy, or ultrasonic energy or that selectively sinks thermal energy.

Tip 162d also has a supplemental transducer 290. In some versions of the invention, supplemental transducer 290 is of the type that can sense whether or not a specific type of tissue is present. For example, it is known that the acoustic reflectivity characteristics of certain tumors are different from the surrounding healthy tissue. For systems used to remove these types of tissue, transducer 324 is an ultrasonic sensor assembly capable of emitting ultrasonic energy and monitoring the reflections. Other transducers 290 are able to sense temperature or impedance.

This version of the system of this invention is used by first using the navigation system to position the catheter so that tip 162d is in the general proximity of the tissue that is to be treated. (Ultrasonic) transducer 290 is then employed to verify that the energy emitting (or sinking) transducer 290 is in close proximity to the tissue to which the pharmaceutical agent should be activated. The signals from transducer 290 can be presented on display 82.

A further benefit of this version of the system is that transducer 290 provides a final verification that the energy emitting (or sinking) transducer 288 is where it should be prior to activation of the transducer 288.

FIG. 27 illustrates another catheter tip 162e of this invention. In addition to the magnetic transducers (navigation markers) 166 and 168 and transducer 288, catheter tip 162e includes a transducer 294 used to determine if a specific chemical reaction has occurred. More particularly, the type of reaction transducer is used to detect is one that would indicate the release of the triggering of the agent is having the desired therapeutic effect.

Thus, in some versions of the invention, transducer 294 may comprise a light source and a lens for capturing detected, reflected light. The reflected light is focused by the lens and returned by a fiber optic cable internal to the catheter to an analyzer. One such analyzer capable is a Raman spectrograph. This particular type of device determines the extent the Stokes Raman scattering of the illuminated molecule. The Stokes Raman scattering is highly specific for particular types of molecules.

Thus, in this version of the invention, during or after the emission (or absorption) of energy to trigger the desired agent, the signals from transducer 294 are analyzed to detect the presence/absence of molecules that should be present/absent if the desired chemical reactions are occurring. This almost real time analysis of the effects of the trigging of the agent allows the practitioner to determine if the desired therapeutic action has occurred. If the desired therapeutic action has not occurred, the practitioner can then adjust the procedure as may be necessary.

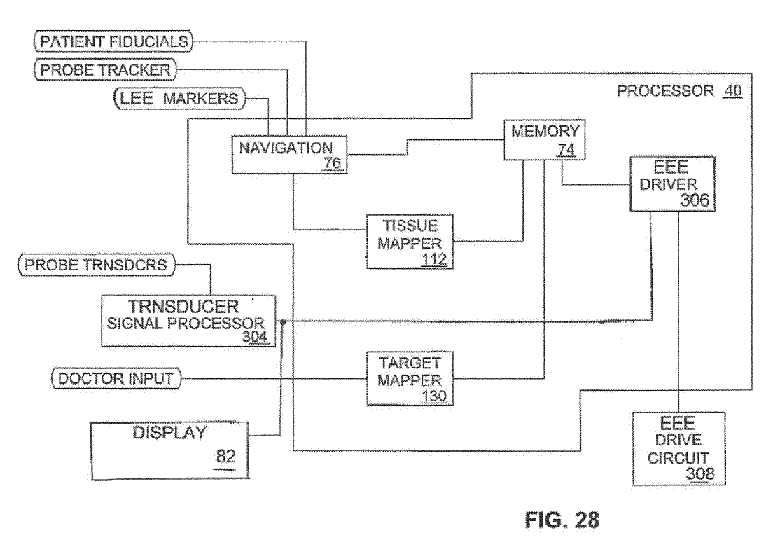

FIG. 28 depicts the architecture of processor of the system of this invention when one of the above described transducers 290 or 294 is incorporated into the catheter tip or probe to which the energy emitting element (EEE) is mounted. Not shown in FIG. 28 is the reference image or data the doctor uses to target the tissue that is selected for treatment.

The signal from the transducer (probe transducer in FIG. 28) is applied to a processor 304. The output form this signal is the circuit that drives the energy emitting elements, driver 306. The output from processor 304 is also provided to a display device that allows the practitioner to see if the sensor indicates that the target tissue is present. (In the case of sensor 294, the data presented indicates whether or not the desired chemical reaction is occurring.) In FIG. 28, this device is display 82.

If the transducer is of the target sensing variety, transducer 290, upon the EEE driver 306 determining that the EEE probe is adjacent the target tissue, the driver then uses the sensor signal as a backup determination of the location of the probe. If this determination is affirmative, the EEE driver 306 then asserts the signals that cause the EEE drive circuit 308 to actuate the EEE 172, 270, 275 or 288. If this secondary determination is negative, the EEE driver 306 informs the practitioner and allows the practitioner to make the determination whether or not the EEE is to be activated.

In an alternative configuration of the above version of the invention, EEE driver 306 employs the signal from the transducer 290 to determine if the EEE probe is in vicinity of the target tissue. The signals from the navigation system are then used to determine if the probe is close enough to actuate the energy emitting element. Again, the practitioner retains the ability to either start or stop the actuation of the energy emitting element.

This version of the invention thus inhibits activation of the energy emitting element unless both the navigation system and the transducer indicate the element is sufficiently close to the target tissue.

If the transducer is of the reaction-sensing variety, sensor 294, the system can be configured to initially actuate the energy emitting element 172, 270, 275 or 288 when it is determined the element is in sufficient proximity to the target tissue. The output from sensor is then analyzed to determine if the desired chemical reaction is occurring. If the desired reaction is occurring, the EEE driver 306 allows the actuation of the element to continue. If the desired reaction is not occurring, or ceases to occur, the driver 306 only allows the element to be actuated, or continued to be actuated, based on a doctor-entered instruction.

The above construction of the invention provides a failsafe that inhibits continued actuation of the energy emitting element unless the desired therapeutic effect of the activation is occurring.

Alternative processes may be used to regulate the amount of light each LEE 36 emits. For example, each LEE 36 may be subjected to a PWM duty cycle that causes the LEE to emit the appropriate amount of light.

Similarly, devices other than LEDs may function as the light emitting elements. In some versions of the invention, there may be a single light emitting unit that emits light over a wide surface. Disposed over this unit is a layer of material the optical transmissivity/opacity of which can be selectively controlled. In this version of the invention, the system regulates the amount of light applied to a specific region of the body by controlling setting the transmissivity/opacity of this screen layer.

Further, it should be appreciated that the generation of the vector of light emitted from each LEE 36, of step 150 is meant to be exemplarily, not limiting. The actual generation of the model of the path of the photons emitted from a particular LEE 36 is a function of dosimetric model of radiation emitted for that particular light emitting element. Thus, many LEEs emit light through space in model that can be considered at conic. The vector generator in the above-described process is the line around which the longitudinal axis of the cone is aligned.

Accordingly, there may be locations spaced from the light blanket where a particular section of tissue could receive photonic energy generated by two or more LEEs 36. Therefore in a variation of step 152, there is a determination of the sum of light from the plural LEEs 36 that intersects each tissue location. Based on this determination, step 154 is performed.

Cooling devices other than Peltier cooling elements 244 may be used to all or selected portions of the tissue immediately below the light blanket or light frame.

It may be desirable to hinge plural light frames 250 together. Likewise the device used to position the energy emitting element and complementary navigation marker in the body may not always be a flexible catheter. In other embodiments of the invention, these two units may be attached to a rigid probe. This probe may be part of an assembly that articulates and/or is able to pivot.

Clearly, other diagnostic devices such as CAT units, X-ray units and navigation units can be used to provide the tissue mapping data.

Further it should be appreciated that this invention may be used when the photosensitive agent is one that does something other than reduce the tissue in which the agent is activated. Thus, the agent can be one that, when activated, causes another desirable therapeutic effect. These effects include, but are not limited to, the generation of more tissue or causing the tissue to output a compound that has its own beneficial effect elsewhere in the body.

Likewise there is no requirement that the system and method of this invention for selectively applying photonic energy to targeted tissue for treatment purposes be limited to procedures in which the light triggers a photosensitive agent. For example some tissue undergoes a desirable transformation solely upon the activation of light at a specific wavelength(s) to that tissue. This invention can be used to ensure that, to a significant extent, the light is applied to the tissue targeted for treatment while the extent to which it is applied to normal tissue is reduced.

Therefore, it is an object of the appended claims to cover all such variations and modifications that come within the true spirit and scope of this invention.

What is claimed is:

1. An assembly for activating a pharmaceutical agent in a living body, said assembly comprising:
an activation unit shaped to be located outside the body, said activation unit shaped to have:
a plurality of individually actuatable energy emitting/sinking components that are directed to the body, the actuation of which results in the transmission of energy through the body, including the skin, that results in the activation of the pharmaceutical agent; and
at least one navigation marker that sends signals to or receives signals from a navigation system;
a navigation unit that receives signals from or sends signals to said activation unit at least one navigation marker and that, based on the signal exchange with the said at least one navigation marker determines the position of each of said activation unit energy emitting/sinking components relative to tissue within the body; and
a processor connected to said navigation unit and to said activation unit, said processor configured to, based on the position of said energy emitting/sinking components relative to the tissue within the body, selectively actuates each said energy emitting/sinking components so as to cause an energy exchange with at least some of the tissue within the body that results in the activation of the pharmaceutical agent in the tissue with which there is an energy exchange wherein, during the actuation of said energy emitting/sinking components, at least one said energy emitting/sinking component may not be actuated.

2. The assembly for activating a pharmaceutical agent in a living body of claim 1, wherein:
said activation unit includes:
a flexible substrate that, when placed on the body, conforms to the surface of the body, wherein:
said energy emitting/sinking components are located on a surface of the substrate directed to the body; and
a plurality of said navigation markers are located on the substrate; and
said navigation unit is further configured to based on the energy exchange with each said navigation marker, determine the position of said navigation marker relative to the body tissue below said marker and
said processor is further configured to:
for each said activation unit energy emitting/sinking component, based on the position of at least one said navigation proximal to the energy emitting/sinking component, determine the tissue in the body with which said energy emitting/sinking component will exchange energy; and
based on the determine of which tissue the energy emitting/sinking component will exchange energy, selectively actuate the said energy emitting/sinking component.

3. The assembly for activating a pharmaceutical agent in a living body of claim 1, wherein:
said activation unit further includes a plurality of transducers that receive signals from which the type of tissue below said transducers can be determined;
said navigation unit is further configured to, based on the energy exchange with said activation unit at least one navigation marker, determine the locations of said activation unit transducers to body tissue below said activation unit; and
said processor is configured to further regulate the actuation of said activation unit energy emitting/sinking components based on the signals received by said activation unit transducers and the locations of said transducers relative to the body tissue below said activation unit.

4. The assembly for activating a pharmaceutical agent in a living body of claim 1 wherein:
said activation unit, in addition to said energy emitting/sinking elements that activate the pharmaceutical agent, includes a plurality of individually activatable members that cool the body;
said navigation unit is further configured to determine the position of each said cooling member relative to the tissue within the body; and
said processor is further connected to said cooling members to, based on the positions of said cooling members relative to the tissue within the body, selectively and independently activate said cooling members.

5. The assembly for activating a pharmaceutical agent in a living body of claim 1 wherein said activation unit includes a rigid member support to which said energy emitting/sinking components are mounted so that the positions of said components relative to each other and to said at least one navigation marker are fixed.

6. The assembly for activating a pharmaceutical agent in a living body of claim 1 wherein: said energy emitting/sinking components are energy emitters capable of emitting one type of energy from the group consisting of: photonic energy; thermal energy; sonic energy; ultrasonic energy; and RF energy.

7. The assembly for activating a pharmaceutical agent in a living body of claim 1, wherein:
said activation unit at least one navigation marker emits light;
said navigation unit receives the light emitted by said activation unit at least one navigation marker, and based on the received light, determine the position of said navigation marker relative to the body tissue below said marker.

8. The assembly for activating a pharmaceutical agent in a living body of claim 1, wherein:
said activation unit at least one navigation marker generates and emits light;
said navigation unit receives the light emitted by said activation unit at least one navigation marker, and based on the received light, determine the position of said navigation marker relative to the body tissue below said marker.

9. A light blanket, said light blanket including:
a flexible substrate that, when placed over a living body, conforms to the surface of the body, said substrate having a distal side directed to the body and a proximal side directed away from the body;
a plurality of individually actuatable energy emitting/sinking components that are mounted to said substrate and directed to the body, said energy emitting/sinking components configured to emit/sink energy from outside the body into the body so that transmission of energy through the body results in the activation of the pharmaceutical agent; and
a plurality of navigation markers mounted to said substrate that are configured to send or receive signals with a navigation system so that, based on the transmission or reception of the signals by the navigation system, the navigation system is able to determine the position of said navigation markers.

10. The light blanket of claim 9, wherein said energy emitting/sinking components are mounted to the distal side of said substrate.

11. The light blanket of claim 9, wherein said navigation markers are mounted to the proximal side of said substrate.

12. The light blanket of claim 9, further including a foam layer disposed on the distal side of the substrate, the foam layer being formed with openings that open to said energy emitting/sinking components.

13. The light blanket of claim 9, further including a plurality of individually actuatable cooling elements mounted to said substrate that are separate from said energy emitting/sinking components, said cooling elements mounted to said substrate so that, when each said cooling element is actuated said cooling element draws thermal energy from a section of the body below said cooling element.

14. The light blanket of claim 9, wherein said energy emitting/sinking components are energy emitters capable of emitting one type of energy from the group consisting of: photonic energy; thermal energy; sonic energy; ultrasonic energy; and RF energy.

15. The light blanket of claim 9, further including a plurality of transducers separate from said energy emitting/sinking components that receive signals from which the type of tissue below said transducers can be determined.

16. The light blanket of claim 9, further including a plurality of ultrasonic transducers separate from said said energy emitting/sinking components, said ultrasonic transducers configured to transmit sonic energy into and receive reflected sonic energy back from the body.

17. The light blanket of claim 9, wherein said navigation markers emit light.

18. A fiducial marker including:
a base;
means for attaching the base to body section;
a magnetic marker that is removably attached to said base;
a navigation marker separate from said magnetic marker attached to said base for providing an indication of the location of the base using a surgical navigation system.

19. The fiducial marker of claim 18, wherein said navigation marker is a light emitting device that is removably attached to said base.
